(19) United States
(12) Patent Application Publication
(10) Pub. No.: US 2016/0033492 A1
(43) Pub. Date: Feb. 4, 2016

(54) Apparatus for Manipulation and Detection of Magnetic Particles

(71) Applicant: Paul A. Bentley, Eugene, OR (US)

(72) Inventor: Paul A. Bentley, Eugene, OR (US)

(21) Appl. No.: 14/855,161

(22) Filed: Sep. 15, 2015

Related U.S. Application Data
(63) Continuation of application No. PCT/US2014/029974, filed on Mar. 15, 2014.
(60) Provisional application No. 61/799,915, filed on Mar. 15, 2013.

(51) Int. Cl. G01N 33/543

(52) U.S. Cl. CPC G01N 33/5438

(57) ABSTRACT

The present invention pertains to the manipulation and detection of magnetically susceptible components and includes methods of manipulation and detection of magnetically susceptible components and apparatuses for the manipulation and detection of magnetically susceptible components. The apparatuses and systems of the present invention employ controllable magnetic fields to precisely control the orientation, position and relative motion of any magnetically susceptible components within a reaction vessel that is subject to interrogation by a detector system. The methods of the present invention employ controllable magnetic fields to specifically locate magnetically susceptible components in regions of a reaction vessel suitable for interrogation by a detector system.

### CROSS-REFERENCE TO RELATED APPLICATIONS

This application is a continuation of Patent Cooperation Treaty Application No. PCT/US2014/029974, entitled “Apparatus for Manipulation and Detection of Magnetic Particles” filed on Mar. 15, 2014, which claims priority to and the benefit of U.S. Provisional Patent Application No. 61/799,915, entitled “Apparatus for Mixing Reactions that Contain Magnetic Particles, the Separation of the Particles and Detection of the Particles”, filed on Mar. 15, 2013, and the specification and claims thereof are incorporated herein by reference.

### BACKGROUND OF THE INVENTION

The current invention is related to the general areas of synthetic and analytical chemistry, biochemistry, molecular biology and genetics that use apparatuses to manipulate magnetically susceptible components, including functionalized magnetic particles, and detect properties associated with these particles.

### DESCRIPTION OF RELATED ART

The application of magnetism to biological and chemical problems is applicable to techniques such as protein separation, RNA isolation, DNA purification, DNA differentiation, immunochemistry, pathogen detection, molecular imaging, drug delivery, chemical synthesis and decantation. However, present approaches are comparatively inefficient, expensive, have unacceptable environmental impact, lack versatility, and do not provide a suitable means for integrating detection methods with the manipulation methods.

U.S. Pat. No. 8,088,285 (2012) discloses an apparatus for mixing and separating magnetic particles. Notably, the apparatus requires a mechanical means for moving magnets and thereby manipulating the magnetic particles. Additionally, the apparatus does not provide a means for detecting any reaction progress in situ.

U.S. patent application Ser. No. 13/704,258 (2013) discloses a method for detecting a biological target in an affinity assay with the use of a magnetic particle and magnetic force to translate the magnetic particle.

U.S. Pat. No. 8,283,185 B2 (2012) discloses an apparatus for mixing, separating, and localizing a magnetically susceptible polymer within a reaction vessel. Notably, this patent does not disclose a means for detecting any reaction progress within the reaction vessel.

The devices described to date to mix, separate, or position magnetic materials either have a low magnetic intensity, limited repertoire of particle movement, do not possess the capacity to follow reaction progress in situ or lack convenient means for the reaction vessel's removal. Thus the value of alternative magnetic devices that can address these inadequacies in a manner that is economic from both a manufacturing and implementation perspective is high.

### BRIEF SUMMARY OF THE INVENTION

The present invention is directed to apparatuses and methods for the manipulation of magnetic chemicals and the detection of reaction progress associated with a magnetically susceptible component, including, but not limited to functionalized magnetic nanoparticles. The magnetically susceptible components can be adapted to recognize or react with a predetermined biological agent or chemical agent, such as for example predetermined characteristics for cell separation and expansion, predetermined proteins for protein and peptide sample fractionation, predetermined characteristics for cell lysis, organelle isolation, predetermined proteins for capturing biotinylated targets, predetermined proteins for performing protein separation, RNA molecules for performing RNA isolation, DNA molecules for performing DNA purification or differentiation, antigens or antibodies for use in immunochemistry, pathogens for pathogen detection, substrates, reagents, catalysts, other predetermined molecules in molecular imaging and one or more pharmaceutical compounds for use in drug delivery.

One aspect of the invention is apparatus which employs a plurality of magnetic sources with one or more gaps between the plurality of magnetic sources. The plurality of magnetic sources define a cavity with a central axis in which a reaction vessel is located. At least one detector may then be positioned in the one or more gaps between the plurality of magnetic sources. In additional embodiments the plurality of magnetic sources may be permanent magnets or electromagnets wherein the magnetic field produced by each magnetic source independently and substantially defines a primary axis. Various embodiments of the invention employ magnetic sources wherein the primary axis for each magnetic source may be substantially parallel or perpendicular, or in between, to the central axis as defined by the cavity. The exact configuration of magnetic sources and resultant magnetic fields may be varied while still remaining within the scope of the present invention.

Further embodiments envision a plurality of magnetic sources that include permanent magnets or electromagnets and any combination of the two. Depending upon the particular embodiment, each electromagnet may be connected to an independent electrical source which is connected to a controller adapted to control one or more of a plurality of current parameters of the electrical source. In still other embodiments, the electromagnets are coils disposed circumferentially about the central axis.

Another aspect of the invention is a method for manipulating and detecting a functionalized magnetic particle. In preferred embodiments, a plurality of magnetic sources are provided near a cavity wherein there is one or more gaps between the plurality of magnetic sources and the cavity defines a central axis. A reaction vessel containing functionalized magnetic particles is located in the cavity and a detector is positioned in the one or more gaps between the plurality of magnetic sources. Then a means for adjusting the strength of one or more of the magnetic sources is provided. By adjusting the strength of the one or more magnetic sources the functionalized magnetic particles can be translated within the reaction vessel along the central axis. Additional embodiments of this method include adjusting the strength of one or more of the magnetic fields so that the functionalized particles are held substantially within or stationary within one of the gaps between the plurality of magnetic sources. For the purpose of this invention, substantially stationary means that particles are held within the same region of the reaction vessel 16.

Another aspect of the invention is an apparatus for manipulating a functionalized magnetic particle contained within a reaction vessel containing a first and second liquid, wherein the first and second liquid are immiscible. The apparatus is comprised of a plurality of electromagnets near a cavity that defines a central axis. Located in the cavity is a reaction vessel that contains the functionalized magnetic particle. The invention further comprises a controller that is operable for adjusting the magnetic field produced by at least one of the plurality of electromagnets, whereby the controller can selectively translate the functionalized magnetic particle between the first liquid and second liquid.

A further aspect of the invention is an apparatus for manipulating and detecting functionalized magnetic particle contained within a reaction vessel by utilizing a reactive chemical. The reaction vessel has a reactive chemical (e.g. antibody, antigen, DNA, protein, peptide, RNA, cell) attached to the reaction vessel wherein this chemical is capable of associating covalently, or non-covalently with another chemical (magnetic or non-magnetic) in a non-magnetic media (e.g. liquid). In close proximity to this area of the reaction vessel wherein the reactive chemical is attached, there is a means (e.g. giant magnetoresitive sensors) of detecting degree of or changes in magnetic field intensity associated with functionalized magnetic particles binding with the reactive chemical.

Various objects, features, aspects, and advantages of the present invention will become more apparent from the following detailed description of preferred embodiments of the invention, along with the accompanying drawings in which like numerals represent like components.

### BRIEF DESCRIPTION OF THE FIGURES

FIG. 1 is a schematic diagram of an apparatus that is constructed in accordance with one embodiment of the invention.

FIG. 2A is a schematic diagram of one application of the apparatus shown in FIG. 1.

FIG. 2B is a schematic diagram of one application of the apparatus shown in FIG. 1.

FIG. 2C is a schematic diagram of one application of the apparatus shown in FIG. 1.

FIG. 3 is a schematic diagram of one embodiment of the apparatus shown in FIG. 1 which employs an optical detector.

FIG. 4 is a schematic diagram of one embodiment of the apparatus shown in FIG. 1 which employs an optical detector.

FIG. 5 is a schematic diagram of one embodiment of the apparatus shown in FIG. 1 which employs a remote optical detector via fiber optic cable.

FIG. 6 is a schematic diagram of one embodiment of the apparatus shown in FIG. 1 with a reaction vessel that connects to a tap.

FIG. 7 is a schematic diagram of one embodiment of the apparatus shown in FIG. 1 which employs an optical detector.

FIG. 8 is a schematic diagram of one embodiment of the apparatus shown in FIG. 1 with a toroid shaped reaction chamber.

FIG. 9 is a schematic diagram of one embodiment of the apparatus shown in FIG. 1 where the detector employs a frustrated total internal reflection technique.

FIG. 10 is a schematic diagram of an embodiment of the invention wherein the detector is aligned substantially parallel to the longitudinal axis (L).

FIG. 11A is a schematic diagram of an embodiment of the apparatus shown in FIG. 10 wherein the detector sight area is substantially collinear with the central axis.

FIG. 11B is a sectional view along plane A of one application of the apparatus shown in FIG. 11A.

FIG. 12A is a schematic diagram of an embodiment of the apparatus shown in FIG. 10 wherein the detector sight area is substantially offset toward the reaction vessel wall.

FIG. 12B is a sectional view along plane B of one application of the apparatus shown in FIG. 12A.

FIG. 13 is a sectional view along plane A of one application of the apparatus shown in FIG. 11A.

FIG. 14A is a schematic diagram of an apparatus that is constructed in accordance with one aspect of the invention, wherein each of the plurality of magnetic sources produce a magnetic field substantially perpendicular to central axis L.

FIG. 14B is a top down view of the apparatus of FIG. 14A.

FIG. 15 is a schematic diagram of an apparatus that is constructed in accordance with one embodiment of the invention, wherein the plurality of magnetic sources produce a magnetic field substantially perpendicular to central axis L.

FIG. 16A is an alternative embodiment of the apparatus as shown in FIG. 14A.

FIG. 16B is an alternative embodiment of the apparatus as shown in FIG. 14A.

FIG. 17 is a schematic diagram of an apparatus that is constructed in accordance with one embodiment of the invention, wherein the plurality of magnetic sources comprise one or more coils located circumferentially around a magnetically susceptible material.

FIG. 18A is an alternative embodiment of the apparatus as shown in FIG. 1 utilizing reaction vessel surface modification for enhanced detection.

FIG. 18B is close up view of the surface of the embodiment shown in FIG. 18A.

FIG. 19 is a schematic diagram of an embodiment of the invention wherein the detector is located outside a gap between magnetic sources.

FIG. 20 is a schematic diagram of an embodiment of the invention which includes two detectors.

FIG. 21 depicts an embodiment of the apparatus depicted in FIG. 1 containing two or more liquid phases.

### DETAILED DESCRIPTION OF THE INVENTION

The following description of the example embodiments is provided to enable any person skilled in the art to make or use the present invention. Various modifications to these embodiments will be readily apparent to those skilled in the art, and the generic principles defined herein may be applied to other embodiments without departing from the spirit or scope of the invention as set forth in the appended claims. As used herein, the term magnetically reactive denotes a property of a particle through which an external electromagnetic or magnetic field can induce motion of the particle and/or exert a force on the particle. The terms mix, separate, isolate and localize, used in any form herein, should be understood to denote specific examples of the various ways in which a magnetically susceptible particle can be manipulated by an external magnetic field, and such terms are not meant to be exclusive, but rather exemplary in nature. Additionally, the term detector system should be understood to refer to detectors which consist of a source and receiver and detectors which consist of single detector. Furthermore, the term detector system should not be read to include any spatial limitations (i.e. detector and receiver on the same side or on opposite sides). When particular orientations are required by a detector system, they will be separately noted.

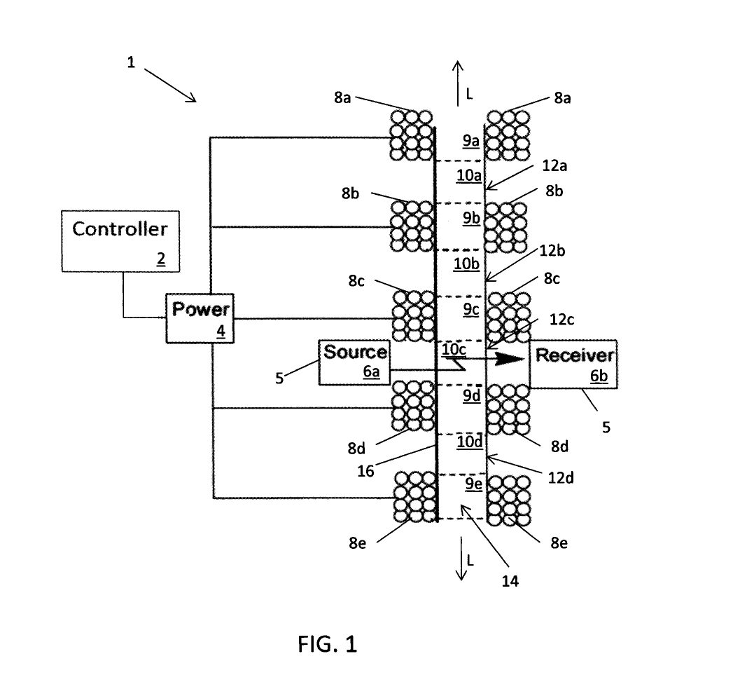

One aspect of the present invention is a device for mixing, separating and/or localizing a magnetically susceptible component within a solution. FIG. 1 depicts a cross-sectional view of a standard configuration 1 comprising a controller 2, a power supply 4, a detector system 5 which may be comprised of a detector source 6A and a detector receiver 6B, a plurality of magnetic sources 8A, 8B, 8C, 8D, 8E, a plurality of gaps between the magnetic sources 12A, 12B, 12C, 12D, a cavity 14, and a reaction vessel 16. The cavity 14 defines a central axis L. Central axis L is defined relative to the cavity 14 which is defined by the plurality of magnetic sources 8A, 8B, 8C, 8D, 8E. The standard configuration 1 encompasses embodiments wherein the reaction vessel 16 can be orientated substantially vertically, substantially horizontally, at a constant angle or at a variable angle relative to the cavity. This makes the standard configuration 1 suitable for use regardless of any prevailing liquid flow, magnetic, electromagnetic, or gravitational field.

In the standard configuration 1 the reaction vessel 16 is substantially round in cross section and the plurality of magnetic sources 8A, 8B, 8C, 8D, 8E are electromagnetic coils which are located substantially circumferentially about the reaction vessel 16. While a round cross section is preferable, the reaction vessel 16 may have any type of cross section including; square, hexagonal, elliptical, etc. The reaction vessel 16 may be made out of any material or materials that are suitable for the liquid contained in the reaction vessel. Example materials include glass, plastic, metal, composite materials or any combination of these or other materials.

The plurality of magnetic sources 8A, 8B, 8C, 8D, 8E, the reaction vessel 16 and the plurality of gaps 12A, 12B, 12C, 12D define a plurality of regions 9A, 9B, 9C, 9D, 9E within the reaction vessel 16. Region 9A corresponds to the region of the cavity 14 within the reaction vessel 16 defined by magnetic source 8A. Region 9B corresponds to the region of the cavity 14 within the reaction vessel 16 defined by magnetic source 8B. Region 9C corresponds to the region of the cavity 14 within the reaction vessel 16 defined by magnetic source 8E. Region 9D corresponds to the region of the cavity 14 within the reaction vessel 16 defined by magnetic source 8D. Region 9E corresponds to the region of the cavity 14 within the reaction vessel 16 defined by magnetic source 8E. Region 10A corresponds to the region of the cavity 14 within the reaction vessel defined by gap 12A. Region 10B corresponds to the region of the cavity 14 within the reaction vessel defined by gap 12B. Region 100 corresponds to the region of the cavity 14 within the reaction vessel defined by gap 12C. Region 10D corresponds to the region of the cavity 14 within the reaction vessel defined by gap 12D.

In the standard configuration 1 the magnetic sources 8A, 8B, 8C, 8D, 8E are electromagnetic coils each of which, when powered, produce a magnetic field within the cavity that is substantially parallel to central axis L. The standard configuration 1 further includes an electrical power source 4 that is capable of supplying independently controllable power to the plurality of magnetic sources 8A, 8B, 8C, 8D, 8E. The power source 4 is connected to the first coil 8A such that in response to a first electrical current passing from the power source 4 through the first coil, the first electrical current generates a first magnetic field substantially parallel to the central axis L. Additionally the power source 4 may be connected to a second coil 8B such that in response to a second electrical current passing from the power source 4 through the second coil, the second electrical current generates a second magnetic field substantially parallel to the central axis L. This general architecture is expandable to any number of coils and magnetic fields.

The standard configuration 1 can be configured according to number of geometries described herein while still embodying aspects of the invention. The coils 8A, 8B, 8C, 8D, 8E, are substantially circumferentially disposed and can become electromagnets with the application of electricity. When powered, each of the coils 8A, 8B, 8C, 8D, 8E produce a magnetic field primarily focused in the regions 9A, 9B, 9C, 9D, and 9E, respectively. Magnetic shielding such as high magnetic permeability metal alloys e.g. permalloy, nanocrystaline grain structure ferromagnetic metal coating and Mumetal (or a material with similar properties to absorb magnetism) may be employed around or in between a coil or coils to shield the magnetic field of that coil from other coils.

Referring to the power source 4 in more detail, the power source 4 provides electrical power, in parallel or in series, to each of the series of coils 8A, 8B, 8C, 8D, 8E. Each of the coils 8A, 8B, 8C, 8D, 8E are configured for generating a magnetic field substantially within a corresponding region 9A, 9B, 9C, 9D, 9E, of the cavity. Each of the series of coils 8A, 8B, 8C, 8D, 8E, can, alternatively be connected to one or more independent power sources, which can be distributed within the power source 4. The standard configuration 1 further includes a controller 2 adapted to control one or more of a plurality of current parameters associated with the electrical power produced by the power source 4. Example controllable current parameters include current magnitude, current frequency, current timing, and current direction. In an alternative embodiment, the controller 4 can be connected to more than one power source 4 and adapted to control one or more of a plurality of current parameters for each power source 4 to which it is connected. The controller 4 can be integrated within the power source 4 or operated as a stand-alone modular component.

Referring to the controller 2 in more detail, the controller 2 is further adapted to control the power source 4 to provide electrical current to each of the coils 8A, 8B, 8C, 8D, 8E in a predetermined pattern. The pattern can be chosen in order to optimize the mixing, separating, detecting, or localizing within the apparatus. The predetermined pattern can include patterns for the provision of electrical current substantially simultaneously or providing current substantially sequentially to each of the coils 8A, 8B, 8C, 8D, 8E. The controller 2 can be any suitable processor, microprocessor, computer, ASIC, integrated circuit, or device that is adapted to produce a predetermined output in response to a set of machine-readable or manual instructions.

The standard configuration 1 includes a reaction vessel 16 disposable all or in part within the cavity 14. The reaction vessel 16 can be of any suitable shape or size for conducting a desired reaction, mixing, or detection. Aspects of the invention envision reaction vessels 16 ranging in size from a lab on a chip or microcapillary applications to a beaker, vat or drum or larger. Additional embodiments can further include an isolating material, such as a copper strip, along the length of the reaction vessel 16 for substantially focusing a magnetic field generated by particular coil 8A, 8B, 8C, 8D, 8E into a particular region 9A, 9B, 9C, 9D, 9E (respectively). Importantly, in some configurations the reaction vessel 16 may not be disposable within the cavity 14. An example of this occurs in an aspect of the invention directed toward lab on a chip applications. In these embodiments, the reaction vessel 16 may be formed in one piece with the larger apparatus, making the reaction vessel 16 co-extensive with the cavity 14. While the term cavity 14 and reaction vessel 16 may refer to different features in certain embodiments, in other embodiments the cavity 14 and reaction vessel 16 may be substantially co-extensive.

The standard configuration 1 further includes a detector system 5 comprised of a detector source 6A and detector receiver 6B. The detector source 6A and detector receiver 6B are positioned in at least one of the gaps 12A, 12B, 12C, 12D, 12E between the plurality of magnetic sources 8A, 8B, 8C, 8D, 8E so that the path in between detector source 6A and detector receiver 6B is not blocked by one of the magnetic sources 8A, 8B, 8C, 8 D, 8E. Providing a clear line of sight between the detector source 6A and detector receiver 6B is desirable for many types of detector systems 5. However, not all detector systems 5 require both a detector source 6A and a detector receiver 6B. Particularly, as in some magnetic detection systems the detection system 5 may solely be a detector receiver 6B. Additionally, some detector systems 5 use light reflection off of a near wall, a far wall, or inside a wall to perform the detector function. In such embodiments, the detector source 6A and detector receiver 6B may be on the same side of the apparatus relative to the reaction vessel 16.

Referring to the detector system 5 in more detail, examples of suitable detector systems include optical systems (e.g. microscope) that detect or employ any wavelength of electromagnetic radiation (IR, UV, etc.), fluorescence detection systems, luminescence detection systems, magnetic detection systems (e.g., by magnetoresistance, Hall effect, coils, AC Susceptometer, superconducting quantum interference device (SQUID), inductive pick up coils), sonic detection systems, electrical detection systems, cold surface plasmon resonance, charge detector via induction or electrostatic methods, ph detectors (hydrogen ion detectors) which may be embedded in the reaction vessel wall 16 in order to contact the liquid within the reaction vessel 16, and particle detectors such as ionization or scintillation detectors. Particular embodiments and particular applications may require a specific type of detector, however embodiments of the invention envision the use of any detection system known to those skilled in the art.

Examples of functionalized magnetic particles which can be manipulated by aspects of the invention include any magnetically susceptible component which is functionalized by biotinylation, the attachment of a small molecule substrate, reagent, catalyst, the addition of cells, a polymer coating, a silica coating, surface charge, streptavidin, antibody, antigen, DNA, RNA, peptide, protein, or any other suitable functionalization as known by a person skilling in the art. Additionally, nonfunctionalized magnetic particles may also be suitable for use by aspects of the invention, however such particles may typically experience undesirable reactivity with the liquid contained in the reaction vessel 16. The underlying magnetically susceptible component can be any type of material that is reactive to a magnetic or electromagnetic field, including for example particles of iron oxide (II), iron oxide (III) and iron oxide (II, III), nickel, nickel oxide, samarium cobalt, or any other suitable inorganic or organic particle including manganese, lanthanide series elements such as neodymium and erbium, magnetic alloys such as aluminum, nickel, cobalt and copper alloys, metal oxides such as chromium dioxide, cobalt oxide, nickel oxide, or manganese oxide, composite materials such as ferrites or ceramic materials. The magnetically susceptible components may be of any size, from single atoms to the macro scale. In certain embodiments, such as lab on a chip applications, nanometer size magnetically susceptible particles are preferable, while in larger industrial applications, it may be desirable for the magnetically susceptible particles to be many centimeters or larger in diameter.

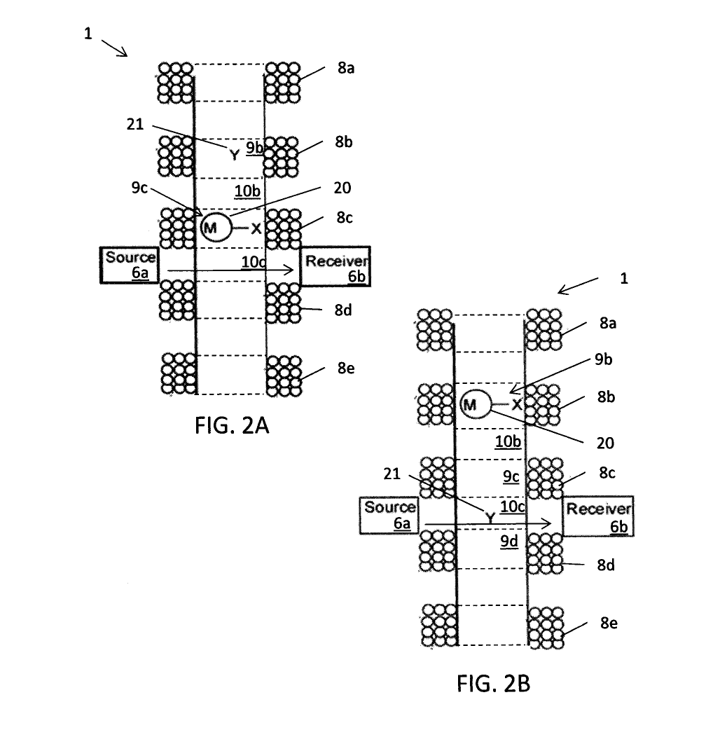

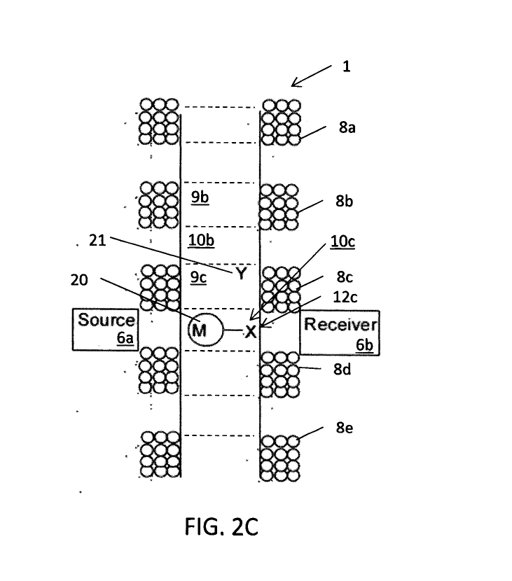

FIGS. 2A, 2B and 2C are schematic diagrams of one application of the standard configuration 1. In FIGS. 2A, 2B, and 2C, for clarity, not all of the components and features depicted in FIG. 1 are shown or labeled. However, it should be understood that FIGS. 2A, 2B and 2C are simply applications of the standard system 1 shown in FIG. 1 and as such, retain the general configuration and functionality of the standard system 1. FIGS. 2A, 2B and 2C depict a standard configuration 1 manipulating a functionalized magnetic particle 20 and an additional substance 21 within a fluid contained in the reaction vessel 16.

Referring to FIG. 2A in more detail, coil 8B can be activated with current running in a first direction that causes a magnetic field to move the functionalized magnetic particle 20 towards to region 9c. Should it be desired that the functionalized magnetic particle 20 be excluded from detection by the detector source 6A and detector receiver 6B which detect primarily in region 100, coil 8C can remain powered. FIG. 2B depicts an application of the standard configuration wherein coil 8B is powered and the resultant magnetic field causes the functionalized magnetic particle to translate to region 9B. The additional substance 21 will be free to translate about the reaction vessel 16.

Additionally, coil 8C can be powered in an alternating pattern with coil 8B. During such operation, the alternating magnetic fields produce a translation of the functionalized magnetic particle 20 for a period of time to region 9C (FIG. 2A) and for a period of time to region 9C (FIG. 2B) and for a period of time to region 10B. The oscillation of the respective magnetic fields generated by coils 8B and 8C causes the functionalized magnetic particle to oscillate between regions 9B, 10B and 9C.

In certain embodiments it may be desirable to utilize the oscillation of a functionalized magnetic particle 20 within different regions to perform mixing operations within the reaction vessel 16. Yet the functionalized magnetic particle 20 may interfere with the detection of an additional substance 21 by a detector system 5 suitable for detecting that additional substance 21. In such an embodiment, a coil 8A, 8B, 8C, 8D, 8E can used to translate the functionalized magnetic particle 20 to a specific region and thus prevent the functionalized magnetic particle from interfering with the detector system 5. Such an application is depicted in FIG. 28. Neither the sequences, nor translocations between regions are limited to those herein described, others will be readily apparent to those skilled in the art and are within the scope of the invention.

Alternatively, should detection of the functionalized magnetic particle 20 be desired another sequence of activations and deactivations is necessary. FIG. 2C depicts an application of the standard configuration suitable for detecting the functionalized magnetic particle 20. In FIG. 2C, the power source 4 provides electrical power to coils 8C and 8D. By adjusting the current supplied to coils 8C and 8D, the strength of the magnetic fields produced by coils 8C and 8D may be adjusted. The controller 4 is capable of adjusting the current supplied, and thus the magnetic fields produced by coils 8C and 8D such that the functionalized magnetic particle 20 is translated to and maintained in region 100. The detector system 5 located in gap 12C has a view of the reaction vessel in region 100. Once the magnetic particle 20 is translated to region 10C, a detector system 5 adapted to detect the functionalized magnetic particle 20 can detect the presence of the functionalized magnetic particle 20 in region 100.

The application shown in FIG. 2C has numerous advantages over existing methods. Importantly, the concentration of the functionalized magnetic particle 20 in region 100 is artificially increased relative to the concentration in the reaction vessel 16 as a whole. Additionally, by locating the detector system 5 in a gap 12C between magnetic sources 8C, 8D, the apparatus is capable of controllably translating the functionalized magnetic particles 20 in either direction along the central axis L.

Referring to the detector system 5 in FIG. 2C in more detail, it is important to note that the use of a single detector source 6A and detector receiver 6B is exemplary in nature and is not intended to be limiting. Alternative embodiments of the standard configuration 1 contemplate a plurality of different detector systems 5 positioned in the plurality of gaps 12A, 12B, 12C, 12D, between the plurality of magnetic sources 8A, 8B, 8C, 8D, 8E. The plurality of detector systems 5 may be coordinated in a variety of ways. For example, each detector system 5 may only be capable of detection when the component for detection is in the appropriate position with the appropriate selectivity. Alternatively, each detection system 5 may utilize a different detection method. Still further, the different detection systems 5 may employ the same detection methods but have different levels of sensitivity. Additionally, it is important to note that the interrogation by a detector system may coincide with a specific particle passing across the path of the detector system 5, a specific particle being held substantially stationary in the field of view of the detector system 5, or the with specific magnetic particles being excluded from the path of the detector.

Additionally, the apparatus shown in FIG. 2A is capable of using inductive heating to selectively heating the functionalized magnetic particle 20 within region 9C inside the reaction vessel 16 without translating the functionalized magnetic particle 20 along axis L. Inductive heating will take place when the magnetic field is oscillated rapidly through the functionalized magnetic particle. When the magnetic field created by coil 8C is oscillated by the control 2 on the order of 25 kHz, or 1000 kHz or more, the magnetic particles heat up. Notably the functionalized magnetic particle 20 heats first, and then the bulk liquid in the reaction vessel 16 heats. Localized heating may be desirable when, for example, the additional substance 21 may be damaged by the amount of heat necessary to make the functionalized magnetic particle 20 reactive. The most effective frequency for heating a particular functionalized magnetic particle will vary by particle. However, the apparatus shown in FIG. 2A is capable of producing oscillating magnetic fields with a wide range of frequencies. This makes the apparatus particularly suitable for inductive heating.

Inductive heating as described in the previous paragraph may be combined with the translation of a functionalized magnetic particle 20 through a reaction vessel 16. A combination of inductive heating and translation offers previously unavailable opportunities to streamline chemical production processes into a single step. Notably, inductive heating via an oscillating magnetic field may take place while the functionalized magnetic particles are being subject to a net attractive or repulsive magnetic field. This enables the translation, or mixing, of functionalized magnetic particles between different regions while the functionalized magnetic particles are subject to inductive heating.

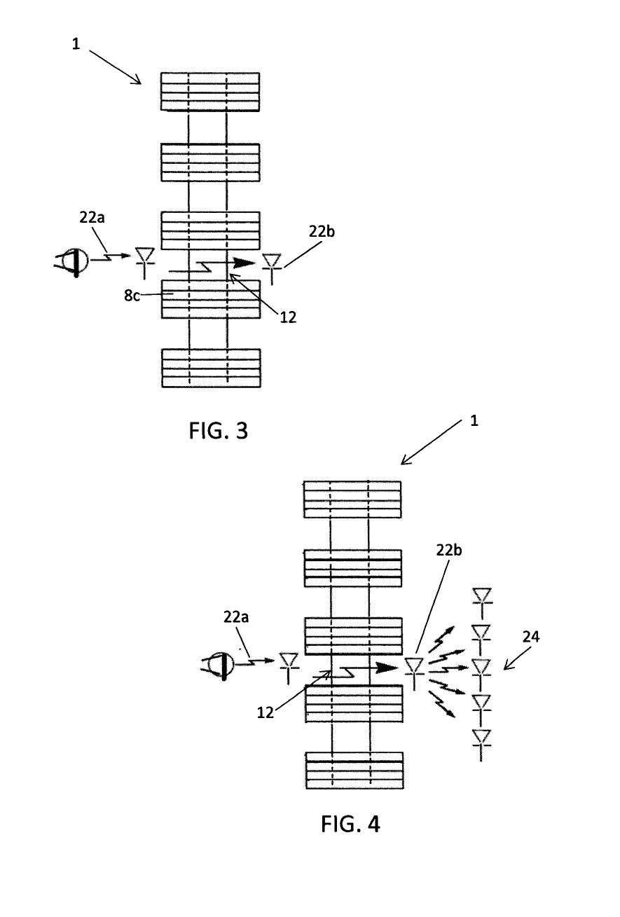

FIGS. 3, 4, 5, 6, 7, 8 and 9 depict variations of the standard configuration 1 shown in FIG. 1, all of which embody aspects of the invention. In FIGS. 3, 4, 5, 6, 7, 8 and 9, for clarity, not all of the components and features depicted in FIG. 1 are shown or labeled. However, it should be understood that FIGS. 3, 4, 5, 6, 7, 8 and 9, are simply various embodiments of the standard system 1 shown in FIG. 1 and as such, retain the general configuration and functionality of the standard system 1.

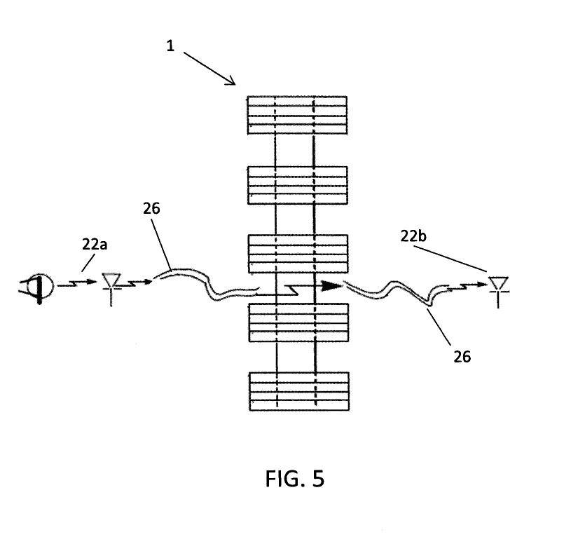

FIG. 3 depicts a standard configuration 1 that employs an optical detector system. Such an embodiment utilizes a detector system 5 comprising a photodetector 22B such as a monochromatic photodiode opposite to a light source 22A. As the embodiment shown in FIG. 4 demonstrates, the arrangement can be extended to disseminate the photo detector 22B signal to an array of diodes 24. Alternatively, as showing in FIG. 5 elements of the detector system 5 maybe positioned remotely from the plurality of magnetic sources 8A, 8B, 8C, 8D, 8E via a suitable means such as fiber optic cables 26 or mirrors (not shown).

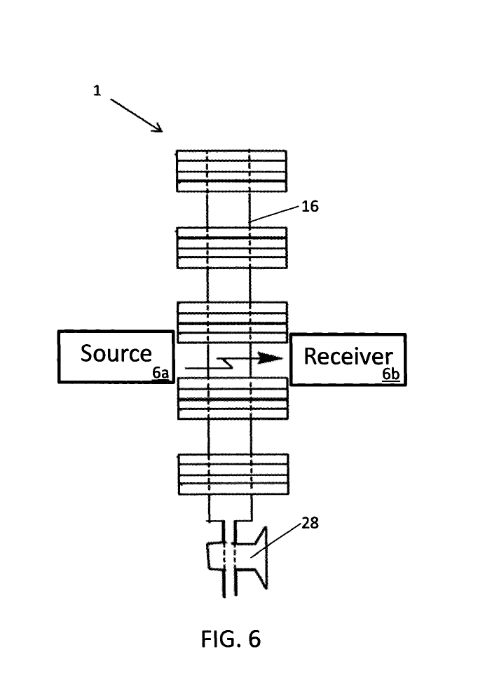

In certain applications it may be desirable to have the option to permit the removal of fluid from the reaction vessel 16 under gravity or pressure from gas or additional liquid via a tap 28, as shown in FIG. 6. In the embodiment shown in FIG. 6 magnetic particles (not shown) may be retained and during any sort of fluid removal by powering one or more of the plurality of magnetic sources 8A, 8B, 8C, 8D, 8E such that the magnetic particles (not shown) are held substantially in place while fluid is removed via the tap 28.

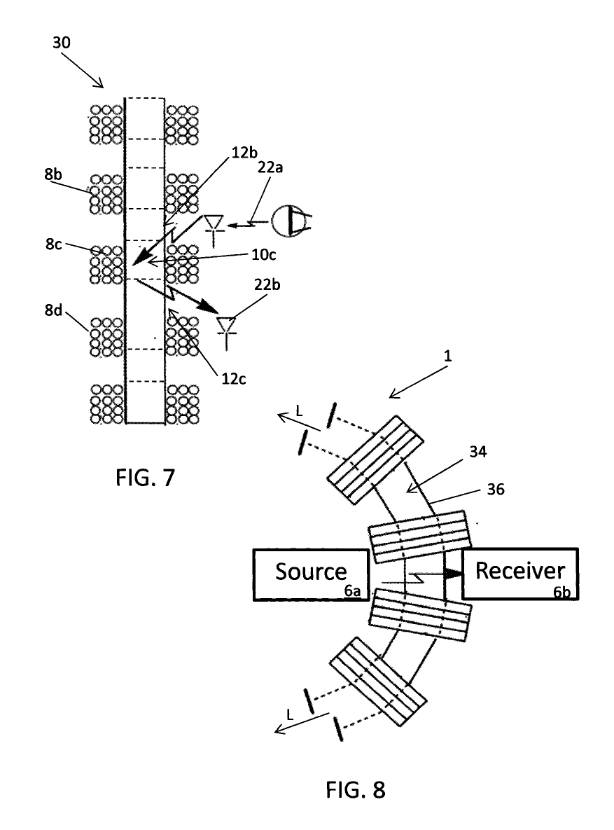

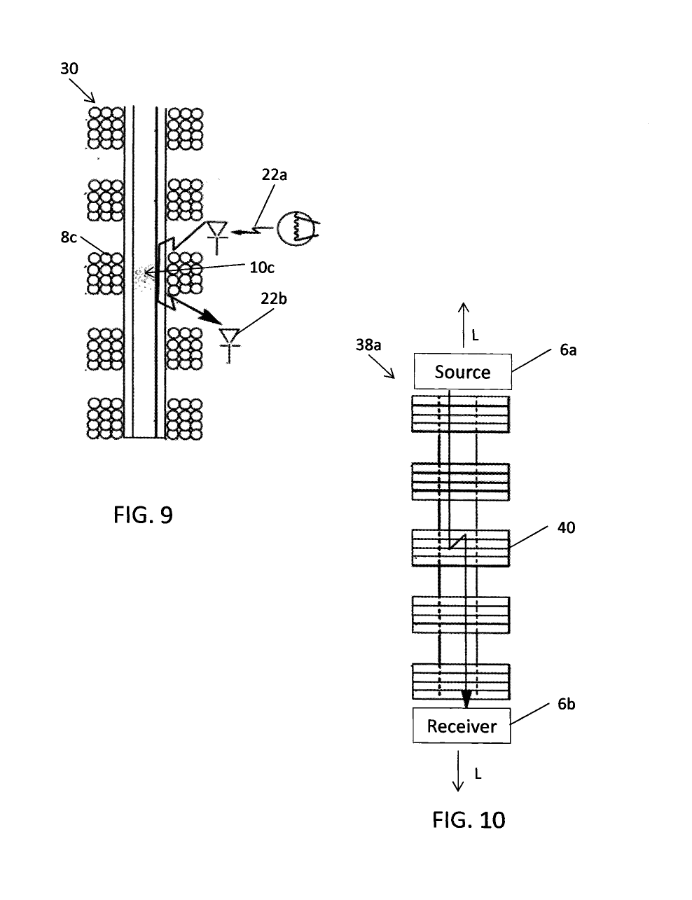

Another embodiment of the standard configuration 1 is shown in FIG. 7. In this embodiment the light source 22A produces light that travels through from a gap 12B between the magnetic sources 8B, 8C. The light produced by the light source 22A is directed towards the internal surface on the far side of the reaction vessel 16 within cavity 9C as defined by a coil 8E. If the internal surface is sufficiently reflective with an appropriate angle the detector path will reflect back toward a photodetector 22B and exit the electromagnet through a gap 12C between a different set of magnetic sources 8C, 8D. Another embodiment which uses frustrated total internal reflection is shown in FIG. 9. In the embodiment in FIG. 9 the light from the light source 22A employs optical frustrated total internal reflection within the reaction vessel wall. The internal reflection could take place along the area of a coil, as in FIG. 9, or between a gap in UNO coils. The light from the light source 22A is reflected within the wall of the reaction vessel 16. At a location where particles are located on the reaction vessel wall 16 the light will experience frustrated internal reflection and be scattered outside of the reaction vessel 16. A photodetector 22B may be used to detect the scattered light.

The variety of detector methodologies are equally applicable to scenarios where the reaction vessel 16 adopts a toroid shape, such as in FIG. 8.

In other embodiments, the standard configuration 1 can include a temperature control means (not shown) disposed or connected to one or more of the cavity 14, the reaction vessel 16, and the electromagnets 8A, 8B, 8C, 8D, 8E. The temperature control means functions to maintain a suitable reaction temperature in the reaction vessel 16 while electrical current passes through the coils 8A, 8B, 8C, 8D, 8E and protect the coils 8A, 8B, 8C, 8D, 8E from damage whilst allowing coils 8A, 8B, 8C, 8D, 8E to produce an optimal magnetic field. For the coils 8A, 8B, 8C, 8D, 8E, the temperature control means can include an internal or external core and/or wrap that insulates the cavity 14 from any heat generated by the coils 8A, 8B, 8C, 8D, 8E when one or more of the coils 8A, 8B, 8C, 8D, 8E is conducting electricity. Alternatively, the temperature control means for the coils 8A, 8B, 8C, 8D, 8E can include both heating and cooling materials, such as a heat source and a heat sink, that are adjustable to maintain a specified temperature. For the cavity 14, the temperature control means can include for example a fluid bath within which the cavity 14 is submersible, a cooling or heating jacket disposed around the cavity 14, a cooling or heat exchanging apparatus operable as a heat sink, or any other suitable means or mechanism for removing heat caused by the coils 8A, 8B, 8C, 8D, 8E away from the reaction within the cavity 14 and/or reaction vessel 16.

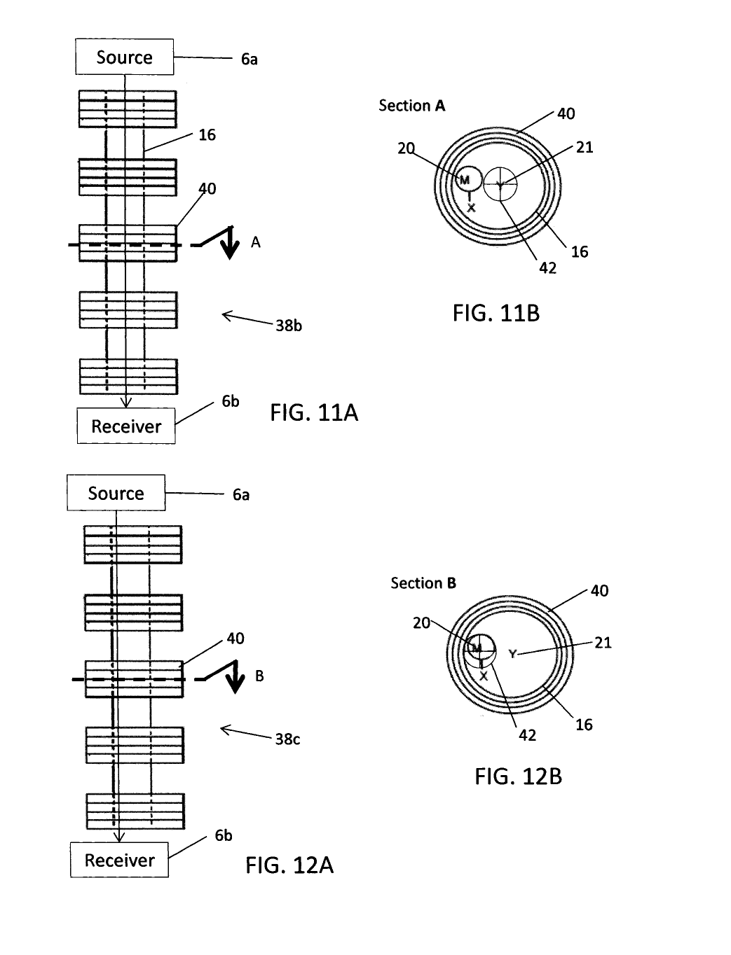

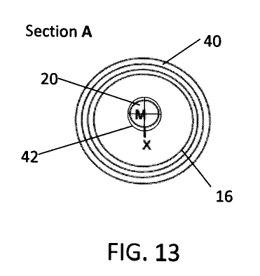

Another embodiment, as shown in FIG. 10, is where the detector system 5 employs a detector sight area 42 substantially parallel to the central axis L of the cavity 14. FIG. 11A depicts an embodiment of FIG. 10 where the detector sight area 42 is substantially co-linear with central axis L. Upon activation of the coil 40, a functionalized magnetic particle 20 contained in the reaction vessel 16 will translate to the edge of the reaction vessel 16. This end result of this movement is shown in FIG. 11B. When the detector sight area 42 is positioned co-linear with central axis L the detector may detect exclusively the non-magnetic particles if the non-magnetic particles and the detector are compatible. Alternatively, as shown in FIG. 12A, the detector system 5 may be offset relative to axis L such that the detector sight area 42 may be located substantially toward the edge of the reaction vessel 16. As shown in FIG. 12B, if the detector sight path 42 is positioned substantially towards the edge of the reaction vessel 16 the detector system 5 will permit detection of a functionalized magnetic particle 20, if the functionalized magnetic particle's 20 functionality and the detector are compatible. Upon deactivation of the coil 40, a functionalized magnetic particle 20 will not remain around the edge but will also translocate to the center of the reaction vessel 16. Thus, as shown in FIG. 13, when the detector sight area 42 is positioned substantially in the center of the cavity, upon coil 40 becoming unpowered a functionalized magnetic particle 20 which was previously undetectable (as in FIG. 11B) may become detectable if the detector system 5 and the functionalized magnetic particle 20 are compatible.

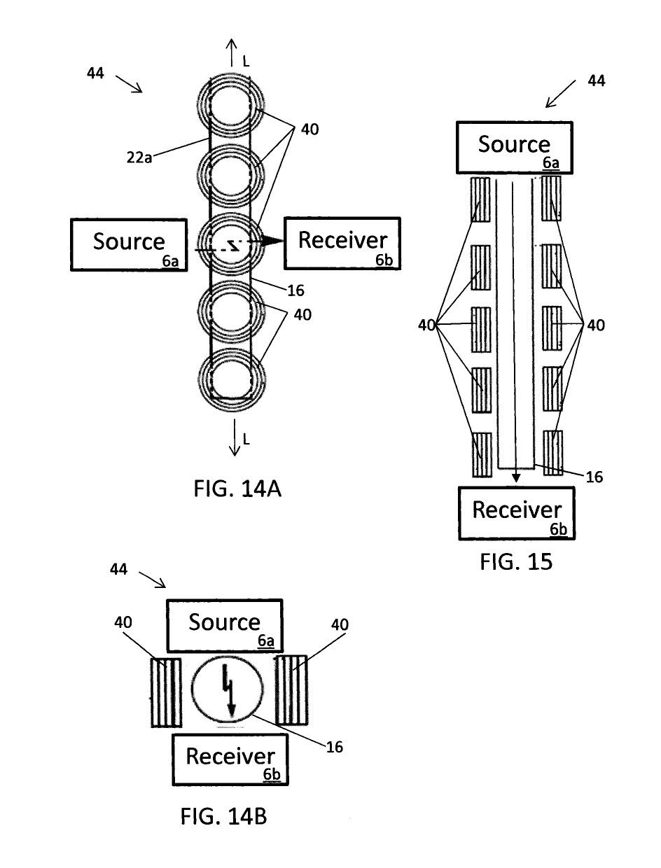

Another embodiment which is a departure from the arrangement of electromagnets described thus far is a perpendicular electromagnet configuration 44 as shown in FIG. 14A. In such a configuration a plurality of electromagnet coils 40 are located with their axes primarily perpendicular to the central axis L. As a result, the magnetic field produced by the electromagnet coils 40 within the cavity 14 and reaction vessel 16 are substantially perpendicular to the longitudinal axis of the cavity. Notably, the embodiment shown in FIG. 14A is suitable for use either with a straight or toroid shaped reaction vessel 16. As shown in FIG. 14B, which is a top down view of the embodiment show in FIG. 14A, the use of electromagnets as shown in FIG. 14A allows the detector source 6A and detector receiver 6B to function at the same location relative to the central axis L of the reaction vessel 16 as the coils 40. Additionally, the detector source 6A and detector receiver can have a field of view co-linear with the axis of the coils 40. The use of electromagnets 40 that produce fields substantially perpendicular to the central axis L can be employed in isolation as depicted or as combination with other features taught in other embodiments. An illustrative embodiment, such as the one shown in FIG. 15, has two sets (defined as a group of coils in line relative to the central axis L) of coils 40 located on opposite sides of the reaction chamber 16.

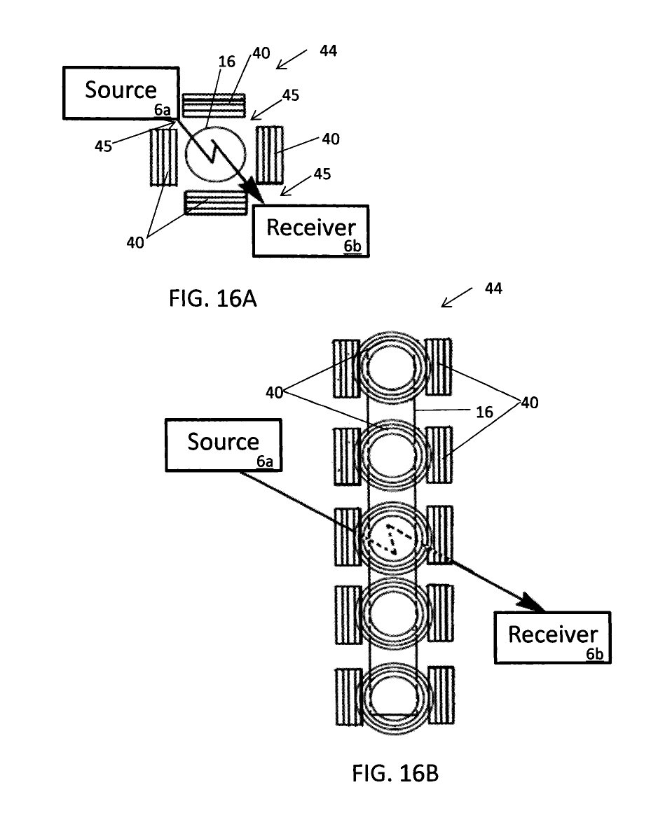

Additional embodiments include perpendicular electromagnet configurations 44 where 4 sets of coils 40 are presented with the coil 40 axis primarily perpendicular to the reaction vessel 16 central axis L, such as shown in a top down view in FIG. 16A, and as shown in a side view in FIG. 16B. In the embodiment depicted in FIGS. 16A and 16B, the detector source 6A and detector receiver 6B use a gap 45 between the plurality of coils 40 to maintain a line of sight through the cavity 14 and reaction vessel 16.

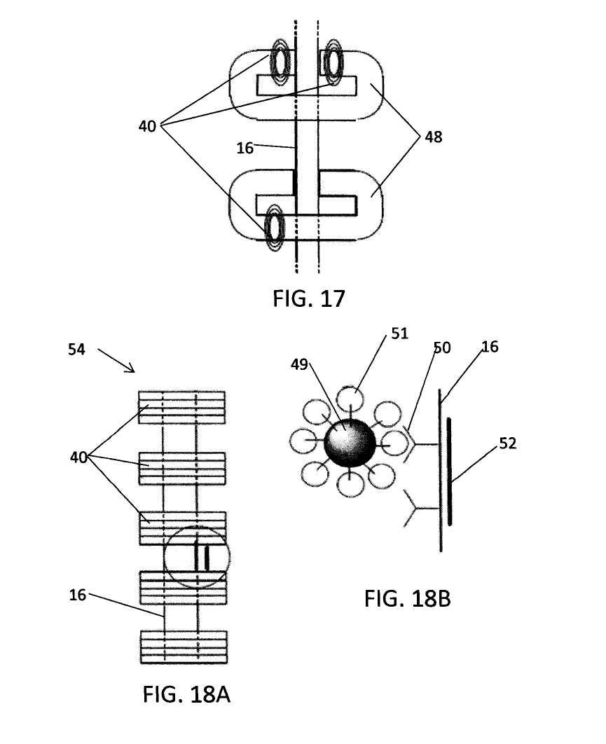

Another embodiment of the invention is shown in FIG. 17. In this embodiment an electromagnet with an axis primarily perpendicular to the reaction vessel is achieved by coils 40 arranged circumferentially around magnetically core 48 that is located circumferentially around the reaction vessel 16. The magnetic core 48 is constructed from a magnetically susceptible material such as iron. The use of a magnetic core 48 provides additional control of the shape of the magnetic field produced by the controller 2 powering the coils 40. The particular magnetic fields created by the various arrangements of magnetic sources in the various described embodiments are intended to be exemplary in nature. The preferred magnetic field will vary depending upon the needs of a person practicing the invention. However, a person of ordinary skill in the art will recognize the invention to cover all combinations of electromagnetic fields that can be produced by a plurality of electromagnetic sources positioned perpendicular and parallel and in between, relative to the central axis L.

Another embodiment is where the detection properties of an apparatus such as shown in FIG. 1 are enhanced with a first component 51 (e.g. Antigen, DNA, RNA, cell, protein, peptide) attached to a material/chemical that can be detected 49 (e.g. magnetic bead with an additional component with for example, but not limited to fluorescent functionality or optically sensitive functionality for techniques including, but not limited to optical frustrated total internal reflection) in solution and a second component 50 attached to reaction vessel 16 wall via e.g. terminal olefin or silane, that can be recognized by the first component 51. FIG. 18A depicts such an embodiment, with FIG. 18B showing a more detailed view of the reaction vessel 16 wall. The recognition reaction between the first component 51 and second component 50 may be covalent or non-covalent. Thus the material/chemical that can be detected is brought into closer proximity to the detector 52 by associating to the second component 50 on the reaction vessel surface (direct ligand quantification). In other cases, The second component 50 maybe not attached to the reaction vessel, but instead attached to another material/chemical e.g. bead suspended in the liquid. Such approaches may compliment AC Susceptometer detection among other detectors. Alternatively a competitive inhibition indirect method could be employed where the ligand to be indirectly quantified is introduced to the solution, and binds to the complimentary component on the reaction vessel wall. The surfaced is washed, or unbound magnetic components are translocated away from that zone by the selective powering of a remote coil 40. Then the same ligand with the material/chemical that can be detected attached is added in solution to the complimentary chemical sites, on the reaction vessel 16 surface, that are still free can bind the ligand with the material/chemical that can be detected. Thus the material/chemical that can be detected has been brought closer on average to the detector 52 for a more sensitive response.

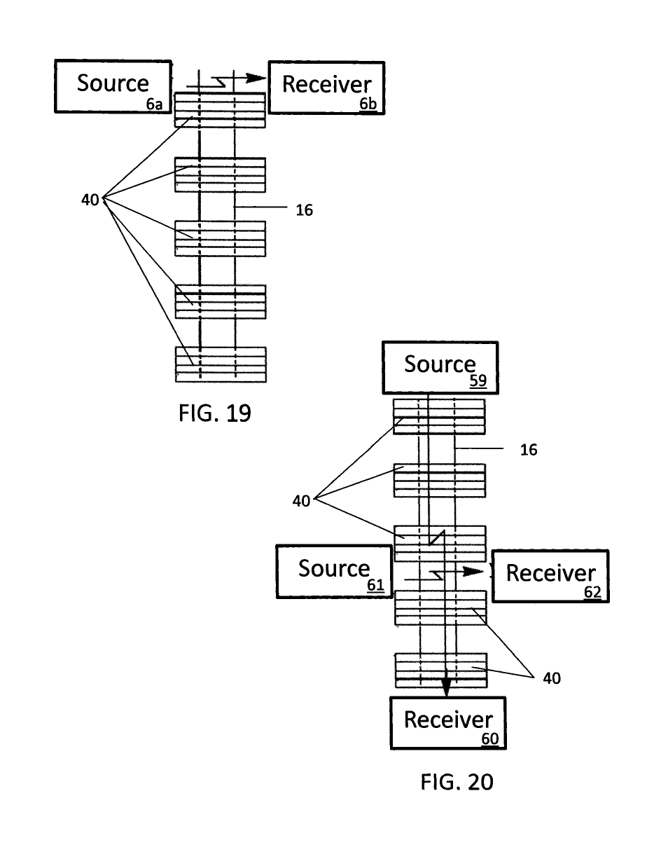

FIG. 19 discloses an embodiment employing a detector source 6A and detector receiver 6B where the line of sight between the detector source 6A and detector receiver 6B is not within the array of coils, not between a coil, nor between a gap between coils, but through the cavity 14 and reaction vessel 16 prior to the a first or last coil.

Combinations of two or more embodiments described above are possible and are embraced by the invention. FIG. 20 discloses an embodiment comprising a first detector system comprised of a first detector source 59, a first detector receiver 60, and a second detector system comprised of a second detector source 61 and a second detector receiver 62. In FIG. 20 the two detector paths are both perpendicular and parallel to the longitudinal axis of the cavity. Additionally, the first and second detector systems 5 may be different types of detectors so that multiple properties of the fluid contained in the reaction vessel 16 can be detected.

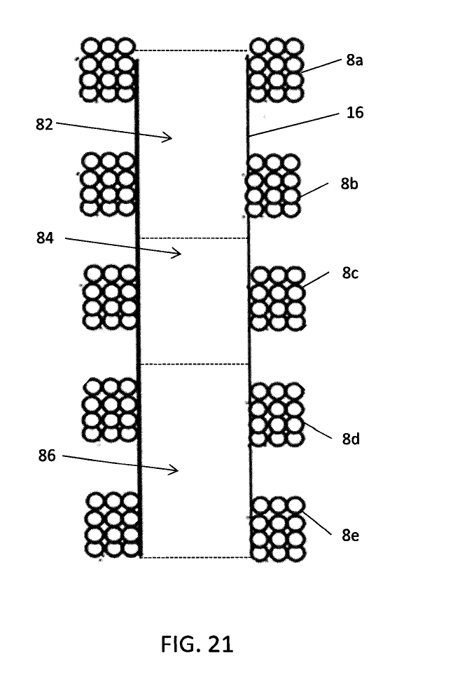

An additional aspect of the invention is the ability to perform multiple step reactions in a single reaction vessel to derive different products without removal or addition of chemicals, nor change in temperatures. The reactions may include, but are not limited to chemoselective, regioselective, diastereoselective and enantioselective reactions. A combination of reactions may constitute an orthogonal protecting group strategy. A combination of reactions may constitute protection strategic functional group transformation (e.g. coupling in the case of peptide or carbohydrate) deprotection and cleavage from the magnetic component. Depending on the substrate at the start of the reaction sequence and the order the sequence is carried, dictated by the order that the substrate with magnetic component is exposed to different immiscible liquid layers. Each liquid layer predominantly contains chemicals associated with at least a single reaction upon the substrate. The sequence, of reactions is thus dictated by the sequence of translations that the magnetic sources induce in the magnetic component. Conventional approaches often suffer from the reagent or catalysts of one step being incompatible with the reagent of catalyst of another step, by them reacting together in an undesired manner. Where the two incompatible reagents or catalysts are miscible in the same liquid layer this is a significant problem. However should the incompatible reagents or catalysts be separated by a liquid, liquid phase barrier this problem is significantly reduced. Another third layer may exist between the two liquid layers providing the magnetic component can pass through this additional layer. FIG. 21 shows an embodiment of the apparatus as shown FIG. 1. For clarity, not all of the components and features depicted in FIG. 1 are shown or labeled. However, it should be understood that FIG. 21 is an embodiment the standard system 1 shown in FIG. 1 and as such, retains the general configuration and functionality of the standard system 1.

The embodiment shown in FIG. 21 is adapted to perform multiple step reactions without the addition or removal of liquid from the reaction vessel 16. Contained within the reaction vessel 16 there are a first region 82, a second region 84 and a third region 86. The liquids contained in adjacent regions are immiscible. Thus the liquid in the first region 82, is immiscible with the liquid contained within the second region 84 and the liquid in the second region 82 is immiscible with the liquid in the third region 86. Notably, the liquid in the first region 82 and the liquid in the second region 86 may be immiscible or not. The embodiment shown in FIG. 21 is applicable to all combinations of immiscible liquids. Additionally, some embodiments will only have a first and a second region containing two immiscible liquids. The general approach wherein adjacent regions contain immiscible liquids is applicable to both cylindrical reaction vessels and toroidal reaction vessels. A toroid shaped reaction vessel is preferable in situations where a first and third liquid in different regions are identical but need to remain separated by a different liquid. In such a case, in a vertically oriented reaction chamber the first liquid (if it is located highest in the reaction vessel) may pass through the different liquid as globules depending on the density and viscosity of the first and third liquid and the different liquid. However, a toroid shaped reaction vessel enables the physical separation of the first and third liquids. Thus, in a toroidal reaction vessel, two of the liquid layers may be miscible, but are physically separated at either side of the toroid. Furthermore, a reaction vessel may contain any number of regions of any size.

In operation a functionalized magnetic particle is introduced to the reaction vessel 16. The functionalized magnetic particle can be translated by the activation of particular magnetics sources from the plurality of magnetic sources 8A, 8B, 8C, 8D, 8E through the first, second and third regions 82, 84, 86. The pattern of movement is controlled by the controller 2 and can take on any pattern of movement between and through the different regions. Preferably, the functionalization of the magnetic particle will interact with at least one of the liquids contained in one of the regions.

Another benefit of the embodiment shown in FIG. 21 is that the functionalized magnetic particle can be subject to heating while being held in specific regions. As described earlier, one of the magnetic sources 8A, 8B, 8C, 8D, 8E can be controlled to produce a rapidly oscillating magnetic field. This rapidly oscillating magnetic field can heat magnetically susceptible particles contained within the reaction vessel if the frequency of the field is operable for heating that particle. The exact frequency will depend upon the particular functionalized magnetic particle.

An another embodiment of the aspect of the invention shown in FIG. 21 is a method to realize bands of chemicals attached to the surface of a tube which uses one inert liquid (e.g. fluorinated solvent e.g. perfluorooctane) and a second liquid immiscible with the first liquid (e.g. organic solvent) that contains a chemical to attach (for example, but not limited to antibody, DNA, RNA, peptide) with a portion that will react with the reaction vessel (e.g. tube) surface (terminal olefin, for polystyrene vessel or silane for glass vessel). The inert liquid (e.g. fluorinated solvent e.g. perfluorooctane) layer will mask the reaction vessel surface area you don't wish to attach the chemical (e.g. antibody, DNA) and such a reaction (chemical attachment to vessel surface) will only occur to the area of the vessel exposed to the liquid (e.g. organic solvent) containing the chemical (e.g. antibody, DNA RNA, peptide with a portion that contains a section that will react with the reaction vessel). On completion the liquids are removed and the surface washed. Another chemical can be introduced to a different area of the reaction vessel with repetition of the process with different volumes of liquids.

An exemplary use of the aspect shown in FIG. 21 is a method to reactivate catalysts. Enzymes function optimally with a vicinal layer of water. If there is insufficient water around the enzyme (e.g. in an organic solvent) over a period of time the vicinal water is lost from the enzyme and the enzyme will become inactive. With the enzyme attached to a magnetic component and the appropriate use of controller 2 and a plurality of magnetic sources 8A, 8B, 8C, 8D, 8E the following steps can be implemented. An enzyme which has been damaged by loss of vicinal water layer can be translated from an organic layer into an aqueous layer, mixed within the aqueous layer by appropriate use of controller 2 and one or more of the magnetic sources 8A, 8B, 8C, 8D, 8E, to fully restore the vicinal water on its surface to restore full activity, then again through appropriate use of controller 2 and electromagnets, translate the enzyme back into the organic region to continue catalysis. An example of an enzyme would be lipase in the production of fatty acids methyl esters by transesterification of Triacylglycerols catalyzed by Lipase enzymes. The catalyst may also be inorganic e.g. Palladium that can be reactivated by its movement into the appropriate region/immiscible liquid layer containing the appropriate chemicals.

Another use of the aspect shown in FIG. 21 is a method to improve enantioselectivity in the preparation of a compound containing at least one stereogenic center. With a substrate attached to a magnetically susceptible component and the appropriate use of controller 2 and the plurality of magnetic sources 8A, 8B, 8C, 8D, 8E, the following steps can be implemented. The substrate will be moved into a first liquid region 82 with a first liquid (that may be aqueous e.g. water, organic or fluorous, e.g. fluorinated solvent e.g. perfluorooctane) that contains chemicals e.g. reagents, catalysts that will transform an original functional group of the substrate and thus enantioselectively introduce a stereogenic center and a new functional group. This reaction may be facilitated by mixing with appropriate use of controller 2 and one or more of the plurality of magnetic sources 8A, 8B, 8C, 8D, 8E, within the first liquid region 82. The substrate is then translocated to a second liquid region containing a liquid immiscible with the liquid in the first liquid region. The second liquid region contains chemicals e.g. reagents and catalysts that will transform enantioselectively the undesired enantiomer of the new functional group back to the original functional group. This reaction may be facilitated by mixing within the second liquid region 84 by the appropriate use of controller 2 and one or more of the plurality of magnetic sources 8A, 8B, 8C, 8D, 8E. The substrate is then translocated with appropriate use of controller 2 and one or more of the plurality of magnetic sources 8A, 8B, 8C, 8D, 8E back to the first liquid region 82 and the sequence is repeated causing enantiomeric enrichment. When satisfactory, the substrate is exposed to chemicals to remove it from the magnetic component. An example would be the enantioselective reduction of a ketone to an alcohol. Depending upon the method an additional quench step may be introduced by moving the alcohol substrate into immiscible methanol layer, immiscible with other solvent/liquid layers, with appropriate use of controller 2 and electromagnets. The alcohol would be translocated to a further liquid layer such the third liquid region 86 that contains chemicals e.g. reagents, catalysts that would perform an enantioselective oxidative kinetic resolution upon the alcohol.

Thus, specific apparatuses and methods of using magnetic fields to aid in the detection and manipulation of functionalized magnetic particles have been disclosed. It should be apparent, however, to those skilled in the art that many more modifications besides those already described are possible without departing from the inventive concepts herein. The inventive subject matter, therefore, is not to be restricted except in the spirit of the disclosure. Moreover, in interpreting the disclosure, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

What is claimed is:

1. An apparatus for manipulating and detecting a functionalized magnetic particle comprising:
a plurality of magnetic sources near a cavity wherein there is one or more gaps between the plurality of magnetic sources;
a reaction vessel located in the cavity; and
a first detector positioned in the one or more gaps between the plurality of magnetic sources.

2. The apparatus of claim 1, wherein one of the plurality of magnetic sources is an electromagnet comprising:
a first electrical source connected to a first wire segment such that in response to a first electrical current passing from the first electrical source through the first wire segment, the first electrical current generates a first individually addressable magnetic field.

3. The apparatus of claim 1, wherein the detector is comprised of a detector source and a detector receiver; and wherein the source and the receiver are located on opposite sides of the cavity.

4. The apparatus of claim 1, further comprising:
a second detector positioned in the one or more gaps between the plurality of magnetic sources wherein the first and second detectors have a different functionality.

5. The apparatus of claim 1 wherein the detector is selected from the group consisting of:
an optical detector,
a magnetic field detector,
a sonic detector,
a ph detector,
a charge detector (e.g. induction or electrostatic),
particle detector (e.g. ionization or scintillation), and
an electrical detector.

6. The apparatus of claim 2 further comprising:
a controller connected to the first electrical sources, the controller adapted to control one or more of a plurality of current parameters of the first electrical source.

7. The apparatus of claim 2, wherein the cavity defines a central axis and the first wire segment is a first coil disposed circumferentially about the central axis.

8. The apparatus of claim 2, wherein the cavity defines a central axis, and the first individually addressable magnetic field is oriented substantially perpendicular to the central axis.

9. The apparatus of claim 2, further comprising:
a second electrical source connected to a second wire segment such that in response to a second electrical current passing from a second electrical source through the second wire segment, the second electrical current generates a second individually addressable magnetic field.

10. The apparatus of claim 1, further comprising:
a tap whereby liquid may be removed from the reaction vessel.

11. The apparatus of claim 7, wherein the cavity defines a substantially cylindrical volume.

12. The apparatus of claim 7, wherein the cavity defines a substantially toroidal shaped volume.

13. A method for manipulating and detecting a magnetically susceptible component comprising:
providing a plurality of magnetic sources near a cavity wherein there is one or more gaps between the plurality of magnetic sources and the cavity defines a central axis;
providing a reaction vessel containing said magnetically susceptible component;
providing a detector positioned in the one or more gaps between the plurality of magnetic sources;
providing a means for adjusting the strength of one or more of the magnetic sources; and
adjusting the strength of one or more of the plurality of magnetic sources whereby said magnetically susceptible component is translated within the reaction vessel along said central axis.

14. The method of claim 13 further comprising the step of:
adjusting the strength of one or more of the plurality of magnetic sources whereby said magnetically susceptible component is held substantially stationary in one of the gaps between said magnetic sources.

15. The method of claim 13 further comprising the step of:
providing a temperature control means for adjusting the temperature within the cavity and/or reaction vessel.

16. An apparatus for manipulating a functionalized magnetic particle comprising:
a cavity that defines a central axis;
a plurality of electromagnets near the cavity;
a reaction vessel located in the cavity containing said functionalized magnetic particle;
a first liquid and second liquid disposed within the cavity wherein the first and second liquid are immiscible; and
a controller operable for adjusting the magnetic field produced by at least one of the plurality of electromagnets, whereby the controller can selectively translate the functionalized magnetic particle between the first liquid and second liquid.

17. A method for manipulating a magnetically susceptible component comprising:
providing a plurality of magnetic sources near a cavity wherein there is one or more gaps between the plurality of magnetic sources and the cavity defines a central axis;
providing a reaction vessel containing said magnetically susceptible component;
providing a means for adjusting the strength of one or more of the magnetic sources;
providing a detector positioned in the one or more gaps between the plurality of magnetic sources; and
rapidly oscillating the magnetic field produced by one or more of the plurality of magnetic sources whereby said magnetically susceptible component is inductively heated.
